(19) United States
(12) Patent Application Publication
(10) Pub. No.: US 2011/0009809 A1
(43) Pub. Date: Jan. 13, 2011

(54) EPILATION DEVICE

(76) Inventors: Uwe Bielfeldt, Bad Soden (DE); Pedro Sanchez-Martinez, Kronberg/Taunus (DE); Judith Von Dahlen, Frankfurt/Main (DE); Stefan Sindlinger, Dieburg (DE)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/885,983

(22) Filed: Sep. 20, 2010

Related U.S. Application Data
(63) Continuation of application No. PCT/EP2009/001591, filed on Mar. 6, 2009.

(30) Foreign Application Priority Data
Mar. 20, 2008 (EP) 08 005 323.4

(51) Int. Cl. A61M 37/00; A45D 44/00

(52) U.S. Cl. 604/23; 606/133

(57) ABSTRACT

The invention the concerned with an epilation device for plucking hairs out of a user's skin having a vaporizer unit adapted to vaporize an application material. This allows for cooling the skin by applying a thin coating of application material prior to epilation as the vaporizer is arranged so as to apply the vaporized application material before an epilation head with respect to a use direction (B). Soothing the skin by applying a refreshing application material after epilation if the vaporizer is arranged so as to apply the vaporized application material behind the epilation head with respect to a use direction (B) may also be considered. In some embodiments, the application material is cooled by a cooling unit or heated by a heating unit in order to intensify a cooling or warming effect of the application material.

### CROSS REFERENCE TO RELATED APPLICATIONS

This application is a continuation of prior co-pending International Application No. PCT/EP2009/001591 filed Mar. 6, 2009, designating the United States.

The present invention is concerned with an epilation device and specifically with an epilation device that has means to dispense an application material onto an area of the skin from which hairs shall be plucked.

Epilation devices according to the opening paragraph are known. E.g., German utility model publication DE 94 90 268 U1 discloses an epilation device that has means for moistening the skin by means of a fluid, where the fluid is directly applied to the skin by the means contacting the skin, e.g. via a moistening roller being in contact with the skin.

Patent application FR 2 454 283 describes an epilation device equipped with a manually operated spray to apply a refreshing material.

It is desirable to provide an epilation device that is improved over the known epilation devices.

Such an improved epilation device is provided by the subject-matter of independent claim 1. Embodiments are defined by the dependent claims.

An epilation device as proposed is arranged for plucking out hairs from a user's skin and has a vaporizer unit for vaporizing an application material. The vaporized application material coats the user's skin during regular operation of the epilation device. The cooling effect of the evaporating application material desensitizes the skin and pluckin of hairs is less painful for the user of the epilation device. The vaporizer unit of the proposed epilation device is arranged to dispense vaporized application material into a solid angle area that lies in front of an epilation head with respect to a use direction along which the epilation device is advanced on a user's skin during regular operation. The vaporized application material is then dispensed onto a skin area prior to the epilation process so that the skin area is prepared for the epilation by desensitizing the skin due to a cooling effect. Any other pre-treatment of the skin, e.g. application of heated application material may be added. The application material may also comprise additional substances such skin nurturing substances or coolants such as menthol. In case of liquid application material that evaporates at usual skin temperature, the thin coating of vaporized application material that is dispensed onto the skin evaporates fast and thereby subducts heat energy from the skin. The cooling effect can also refresh or soothe the skin, e.g. if the vaporized application material is dispensed onto the skin after the plucking of hairs. As a vaporized application material is applied in the form of very fine droplets (an aerosol), these droplets can lead to a very thin coating that evaporates fast.

In another embodiment, the epilation device comprises an illumination unit adapted for illumination of the vaporized application material. The illumination visualizes the application of the vaporized application material for the user. It also serves for visual guidance. The illumination unit may be switchable between a first light colour and a second light colour. Two different light colours such as blue and red can indicate to the user whether a cooling effect or a heating effect is employed by the epilation device when dispensing vaporized application material onto the skin.

In a further embodiment, the epilation device has a blower unit and/or a suction unit. These devices can be arranged so that a gas stream is generated that e.g. supports the evaporation of application material coating the skin. The blower unit can be arranged to heat or cool the gas stream before it is directed to the skin to intensify a cooling or heating effect, respectively.

In an even further embodiment, the vaporizer unit is adapted to mix the application material with a gas, e.g. by having a gas stream that releases small quantities of application material from an application material reservoir. When this mixture of gas and vaporized application material is directed to the skin, the gas is e.g. supporting the evaporation of the application material and the cooling of the skin.

In one embodiment, the epilation device has a cooling unit and/or a heating unit for cooling and/or heating of the application material. Applying a cooled application material intensifies the cooling effect. Application of a heated application material supports a soothing effect of the application material.

In another embodiment, the epilation device comprises a container for storing the application material. The container can be arranged to be exchangeable, respectively the container is detachably mounted. This allows e.g. for easy replacement of an empty container or to exchange one container by another container containing a different application material better suitable for the planned epilation procedure.

In an embodiment, the epilation device has a hand piece and a vaporizer station that are both connected by a vapour connector. The vaporizer station houses a vaporizer. The vapour connector is adapted to convey vaporized application material from the vaporizer station to the hand piece. The vapour connector can be detachably mounted to the hand piece so that a user can detach it when the epilation device should be used without applying vaporized application material to the skin.

In a further embodiment, the epilation device has a control unit for selectively controlling the vaporizer unit, e.g. for switching the vaporizer unit on or off or for selectively controlling the amount of application material dispensed per time unit. In an embodiment with a heating unit and/or a cooling unit, the control device can be adapted to control these units and to selectively switch these devices on or off and/or to control the temperature of the application material. In an embodiment with an illumination device and/or a blower/suction unit, the control unit can be adapted to also control these units.

The invention will be further described by reference to figures and by a description of several exemplary embodiments, In the figures

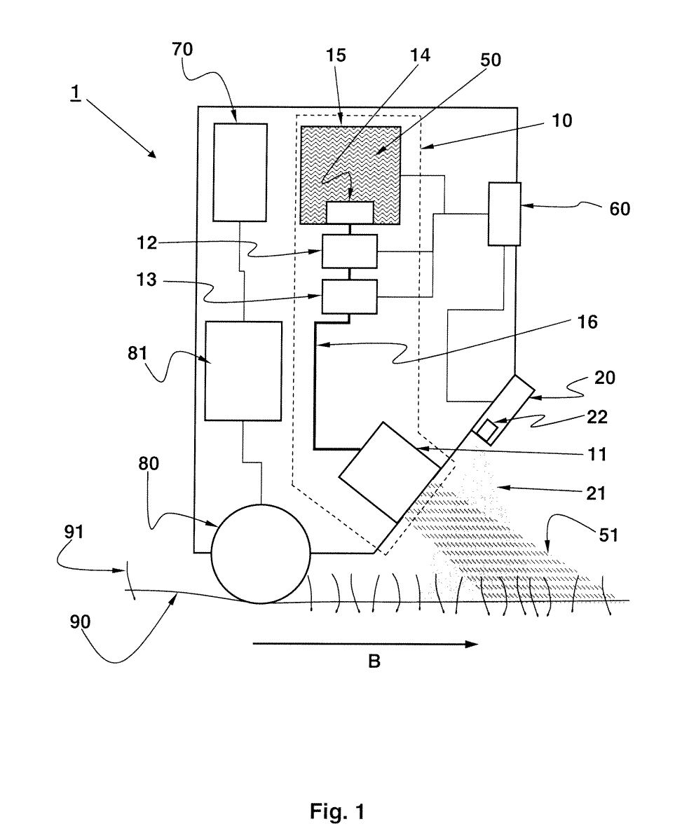

FIG. 1 shows a schematic drawing of an exemplary embodiment of an epilation device as proposed and

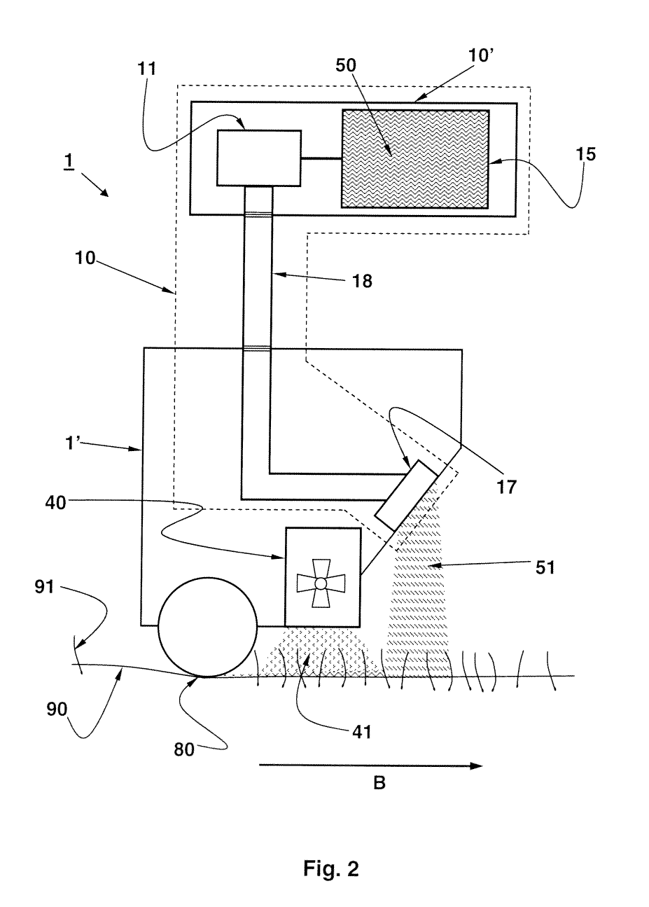

FIG. 2 shows a schematic drawing of another exemplary embodiment of an epilation device as proposed, where the epilation device has a hand piece and a vaporizer station.

FIG. 1 shows a schematic depiction of an exemplary embodiment of an epilation device 1 as proposed. The epilation device 1 has an epilation head 80 that is driven by a motor 81 that in turn is energized by an energy source 70 such as an accumulator. Further, the epilation device 1 has a vaporizer unit 10. The vaporizer unit 10 comprises a vaporizer 11 for vaporizing an application material 50, which is stored in a container 15. By selectively switching a pump 14 on or off, the application material 50 can be conveyed to the vaporizer 11 through a pipe 16. The application material 50 could be a liquid such as water.

A heating unit 12 and a cooling unit 13 are integrated into the pipe 16 for selectively heating and/or cooling the application material 50 before it reaches the vaporizer 11. The heating unit 12 could be arranged as an electric heater such as a continuous flow heater. The cooling unit 13 could be realised by means of a thermoelectric cooler such as a Peltier device (which could also be used to realise a heating unit or a combined heating and cooling unit).

The vaporizer 11, the container 15, the heating unit 12, the cooling unit 13, the pump 14 and the pipe 16 are parts of the vaporizer unit 10 as is indicated by a dashed line in FIG. 1.

During regular operation, the epilation device 1 is advanced along a use direction B on the skin 90 of a user as is schematically shown in FIG. 1. When being operated, the epilation head 80 grips hairs 91 growing on the user's skin 90 and plucks them out. Epilation heads are widely known in the art (e.g. WO 2006/037392 A1 discloses a motor-driven cylindrical epilation head having a plurality of tweezers for gripping hair and plucking it out during rotation of the epilation head) and the present disclosure shall encompass all kinds of realisations. The vaporizer 11 dispenses vaporized application material 51 (e.g. in the form of an aerosol) into a solid angle area that lies in front of the epilation head 80 with respect to the use direction B. The vaporized application material 51 coats the user's skin 90 and the hairs 91 with a layer of fine droplets. The fine droplets evaporate due to the heat energy provided by the user's body and thereby cool the skin area. The cooling effect can be intensified by adding cooling agents such as menthol to the application material 50. Cooling can also be intensified by cooling the application material 50 prior to vaporization with the cooling unit 13.

The application material 50 can be stored in the container 15 at room temperature (e.g. 21 degrees Celsius). which is already below the skin temperature. The cooling unit 13 can cool the application material 50 further down, e.g. to a temperature of 10° C. or a temperature of 0° C. or even below 0° C. The application material 50 may further cool down, depending on the vaporization method, due to an adiabatic expansion during vaporization. Instead of droplets, a fine mist of frozen particles of application material can be applied to the skin. The cooling effect may be used to desensitize the skin nerves so that a plucking pain is less felt during epilation. In an alternate embodiment, the skin 90 is refreshed as the vaporizer 11 dispenses the vaporized application material 51 onto a skin area lying behind the epilation head 80 with respect to the use direction B. Alternatively, also a heating effect may be used. In order to employ a heating effect, the application material 50 is heated by the heating unit 12 prior to the vaporization step. Vaporisation is performed at a temperature below the evaporation temperature of the application material 50. In an exemplary embodiment, the epilation device comprises two vaporisers (or even two vaporiser units) that can apply application material to a skin area lying before or behind the epilation head with respect to the use direction B; one of the vaporizer can then apply cooled application material while the other vaporizer applies heated application material.

In another embodiment (not depicted), the epilation device 1 has two containers 15 containing different application materials that are, e.g., vaporized by a single vaporizer 10 or individually by two vaporizers, each of the two vaporizers being connected with one of the containers. The two application materials could be chosen so as to undergo an endothermic or an exothermal chemical reaction when mixed in order to cool or heat the skin, respectively.

The epilation device 1 according to FIG. 1 has an illumination unit 20 that comprises at least a light source 22 such as an LED. Alight beam or light cone 21 can then illuminate the vaporised application material 51. The illumination unit 20 can be switchable between a first light colour (such as blue) and a second light colour (such as red) to visibly indicate to the user that a cooling effect or a heating effect is employed. respectively. The epilation device 1 also has a control unit 60 to allow the user to selectively switch on or off the vaporizer unit 10 and/or the illumination unit 20 and/or to select whether a cooling effect or a heating effect should be employed.

Alternately or additionally to be energized by a (rechargeable) energy source 70 such as a Lithium-ion accumulator, the epilation device 1 could be energized via a connection to mains voltage (not shown). The energy source 70 and/or the mains voltage is also connected to the various loads present in the epilation device 1 such as the pump 14, the heating unit 12, the cooling unit 13, the vaporizer 10. the illumination device 20, and the control unit 60. The connections are not shown in FIG. 1 for sake of clarity.

Even though the heating unit 12 and the cooling unit 13 are shown as being arranged in the pipe 16 connecting the container 15 and the vaporizer 11, the heating unit 12 and/or the cooling unit 13 can be arranged at any appropriate places in the vaporizer unit 10. E.g. the container 15 and/or the vaporizer 11 could be thermally coupled to the heating unit 12 and/or the cooling unit 13.

FIG. 2 is a schematic depiction of another exemplary embodiment of an epilation device as proposed. The epilation device 1 comprises a hand piece 1′ and a vaporizer station 10′. The hand piece 1′ has an epilation head 80 for gripping and plucking out hairs 91 from a user's skin 90.

The vaporizer station 10′ has a container 15 for storing an application material 50 such as water or an epilation pre-treatment lotion. The container 15 is connected to a vaporizer 11. The container 15 may be realised as a pressure reservoir in which the application material 50 is stored under pressure. The vaporizer 11 can then be realized as a hydraulic spray nozzle. The application material 50 may alternatively be stored in a container together with a propellant stored under pressure as is known from spray cans. The vaporizer 11 can then be realized as a twin-fluid nozzle. Other embodiments are also possible, e.g. the vaporizer 11 could be realized as a mechanical atomizer or an ultrasonic atomizer. The vaporizer station 10′, the vapour connector 18 and the outlet 17 form the vaporizer unit 10 as is indicated by a dashed line in FIG. 2.

During operation, the vaporized application material is conveyed from the vaporizer 11 arranged in the vaporizer station 10′ to the hand piece 1′ via a vapour connector 18 that is adapted for vapour transport such as a flexible tube. Even though FIG. 2 depicts a single-piece vapour connector 18, the vapour connector 18 may be assembled from several pieces. E.g. the vapour connector 18 may be flanged to the hand piece 1′ at an inflow point. The hand piece 1′ has an internally arranged connector through which the vaporized application material is conveyed to an outlet 17 such as a spout. The outlet 17 is arranged for guiding vaporized application material 50 into a solid angle area that is chosen so that a stream of the vaporized application material 51 will coat the skin 90 and the hairs 91 of a user when the epilation device 1 is advanced along a use direction B over a user's skin. In the embodiment according to FIG. 2, the vaporized application material 51 coats a skin area that lies in front of the epilation head 80 with regard to the use direction B. In another embodiment (not shown) the vaporized application material 51 coats a skin area lying behind the epilation head 80 with regard to the use direction B. In even another embodiment (not shown), the vapour connector 18 has an outlet 17 and the vapour connector 18 is arranged so as to be attachable to the hand piece 1′. In this embodiment, an internal connector for conveying the vaporized application material needs not be provided in the hand piece 1′. In general, the vapour connector 18 and/or the outlet 17 may be equipped so as to avoid that aggregated application material is dripping out of the outlet 17.

The epilation device 1 as depicted in FIG. 2 has a blower unit 40 arranged in the hand piece 1′ so as to blow a stream of gas 41 such as air onto the skin that is coated with the vaporized application material. The blower unit 40 could be realized as a motor driven fan. The stream of gas 41 supports the evaporation of application material coating the skin and a fast cooling of the coated skin area is affected. Alternatively, the blower unit 40 could be arranged to release a gas stream 41 from a pressure container through a nozzle. As the gas released from the pressure container undergoes an adiabatic expansion, the gas is cooled and supports the cooling of the skin. Instead of a blower unit a suction unit could be arranged in the epilation device so that a gas stream is generated by suction.

Several of the features shown in FIG. 1 such as the control unit 60 or the heating unit 12 and the cooling unit 13 have not been redrawn in FIG. 2 for sake of clarity. It shall be understood that all obvious combinations of features shown in FIG. 1 and FIG. 2 and features that have only been described shall be encompassed by the subject matter of the present disclosure and that an epilation device as proposed could comprise all the various features and variants.

The container 15 as shown in FIG. 1 and FIG. 2 for storing the application material can be detachably mounted to the epilation device 1 so that different application materials can be used or an empty container can be easily exchanged by another container. Even though it has been described that the application material 50 could be stored under pressure, it is also possible to store the application material 50 without pressure, e.g. the application material 50 can be stored in a flexible bag that is arranged so as to be above the vaporizer during operation. The application material can then be selectively conveyed to the vaporizer 11 by opening or closing a valve and making use of gravitation. The discussed realisations of the vaporizer unit 10 are just exemplary and any other realisations obvious to a skilled person shall be encompassed by the present disclosure.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

1. Epilation device for plucking hairs out of a user's skin having a vaporizer unit adapted to vaporize an application material, wherein the vaporizer unit is arranged to dispense vaporized application material into a solid angle area that lies in front of an epilation head with respect to an use direction (B) along which the epilation device is advanced on a user's skin during operation, such that the cooling effect of the evaporating application material desensitizes the skin.

2. Epilation device according to claim 1 that comprises an illumination unit adapted for illumination of the vaporized application material.

3. Epilation device according to claim 2, wherein the illumination unit is arranged to be switchable between at least a first light colour and a second light colour.

4. Epilation device according to claim 1 that comprises a blower unit and/or a suction unit.

5. Epilation device according to claim 1 wherein the vaporizer unit is adapted for mixing the application material with a gas.

6. Epilation device according to claim 1 that comprises a cooling unit for cooling the application material.

7. Epilation device according o claim 1 that comprises a heating unit for heating the application material.

8. Epilation device according to claim 1 that comprises a container adapted to store the application material.

9. Epilation device according to claim 8, wherein the container is detachably mounted.

10. Epilation device according to claim 1, wherein the epilation device comprises a hand piece and a vaporizer station housing a vaporizer, the hand piece and the vaporizer station being connected by a vapour connector adapted to convey the vaporized application material during operation, the vaporizer station and the vapour connector being parts of the vaporizer unit.

11. Epilation device according to claim 10 wherein the vapour connector is detachably mounted to the hand piece.

12. Epilation device according to claim 1 that has a control unit for controlling the vaporizer unit.
